(19) United States
(12) Patent Application Publication
(10) Pub. No.: US 2025/0281376 A1
(43) Pub. Date: Sep. 11, 2025

(54) Hair Care Composition

(71) Applicant: Joel Marcel CORET, PENNINGTON, NJ (US)

(72) Inventor: Joel Marcel CORET, PENNINGTON, NJ (US)

(21) Appl. No.: 19/075,270

(22) Filed: Mar. 10, 2025

Related U.S. Application Data
(60) Provisional application No. 63/563,545, filed on Mar. 11, 2024.

(51) Int. Cl. A61K 8/25; A61Q 5/02; A61Q 5/12

(52) U.S. Cl. CPC A61K 8/25; A61Q 5/02; A61Q 5/12; A61K 2800/30; A61K 2800/5922

(57) ABSTRACT

Hair conditioning compositions are used to soften hair and to improve its' shine, manageability and to allow it to be styled more easily. Most of these compositions use conditioning agents containing quaternary ammonium chemical groups that adhere strongly to the hair but these have the drawback of presenting an undesirable toxicological profile due to their irritating and biocidal properties which can affect scalp health. It has been discovered that combinations of film-forming agents and certain emollients can advantageously replace the quaternary conditioners to create safe, effective conditioning treatments that present a safer chemical profile, suitable for all hair types. These can be formulated into creams or sprays that can be used in leave-in or rinse off hair conditioning compositions that are described in the invention.

### BACKGROUND

Hair Care products consist of cleansing formulas, generally referred to as shampoos, conditioners and styling aids. The shampoos are mostly based on blends of anionic and amphoteric surfactants that tend to lather during application, creating a pleasant foam which is important to consumer perception of washing efficacy and remove sebum, particles and oils from the scalp and hair surfaces.

Other hair treatments include conditioning products which affect the appearance and behaviour of the hair. They improve shine, reduce combing friction and reduce fly-aways and generally improve styling by affecting the surface properties of the hair fiber. This is achieved by using compounds that deposit on the hair surface and resist rinsing, leaving a surface residue that delivers the desired attributes. The most commonly used conditioning agents are cationic in nature and consist of a permanently charged quaternary ammonium group that interacts with the hair fiber's negatively charged surface; the strong electrostatic interaction ensures the deposition of the conditioning agent on the hair surface. Other components of a conditioning cream may contain of a blend of fatty alcohols, emollients, silicones, polymers and preservatives, as well as fragrance, generally formulated in water. Emolllients and vegetable oils are also used in hair conditioners as these can impart a sifter feeling to the hair; esters and oils and butters are useful in formulations prepared for curly and textured hair where they improve the softness of the hair fiber.

Styling aids may also contain silicones to enhance shine, as well as polymers that form a rigid framework upon drying to physically hold the hair fiber and can contain higher levels of volatile solvents such as alcohols to accelerate drying and setting times. The film-forming polymers used in hair styling preparations form a rigid to semi-rigid network on the hair surface in order to lock the hair in place after drying. Fimformers that form a flexible film upon drying are useful in skin care applications and are used to improve water resistance of sunscreen filters, as well as to improve the wear resistance of pigments in colored formulations.

It has been surprisingly discovered that combinations of flexible film-formers and some emollients can provide a conditioning benefit to hair even in the absence of traditional conditioning compounds such as quaternary surfactants which are contained in most conditioners. There is an significant advantage in the use of conditioners that dispense with these quaternary surfactants because of their inherent irritation profile.

Indeed, the quaternary surfactants are the object of scrutiny due to their high biological impact: these products can be strong irritants as well as biocides: they can affect the dermal layer of the scalp directly if they enter into contact with the living cells of the scalp. The biocidal impact of quaternary surfactants is another concern in the use of this chemistry in hair care: since the scalp comprises a rich microbial ecosystem, commonly referred to as the microbiome, composed of bacterial and fungal organisms. These live in a commensal relationship with the scalp but sudden changes in the equilibrium of the microbiome ecosystem can trigger reactions in the scalp, including irritations and flaking of the scalp. The impact of hair care products on the microbiome can therefore indirectly affect the scalp by displacing this equilibrium.

Whether through direct irritation or through indirect effect on the microbiome, these quaternary compounds could result in potential scalp irritation, excess sebum production and ultimately, in scalp flaking. It is therefore of interest to develop hair care products that can dispense with this chemistry.

### DETAILED DESCRIPTION OF THE INVENTION

It is the object of the current invention to disclose a composition that has been discovered to replace the use of quaternary surfactants in hair conditioners and conditioning treatments. It has been discovered that compositions using a blend of fast-spreading emollients together with flexible film-formers provide a strong conditioning effect on the hair, allowing for soft, shiny, manageable hair to be achieved after topical application of the said formulation, either with a rinsing or when used as a leave-on application to damp hair.

This allows the development of products that treat the hair fiber, providing softness, enhancing manageability and nourishing the hair fiber while being extremely safe toward scalp health.

The use of cationics is ubiquitous in hair conditioners due to the fact that they adhere strongly to the hair surface; this is accomplished due to the strong electrostatic attraction between the negative electric charge present on hair surfaces which attracts the positively charged molecule. Once this molecule adsorbs onto the surface, the fatty component of the cationic surfactant forms a lipid layer on the hair surface which provides lubrification, improving slip and reducing combing friction while also affecting the shine of the hair, by creation of a lipid layer on the surface and by improving fiber alignment. The neutralization of surface charges can also modify the electrostatic properties of the hair by reducing static build-up, improving manageability.

It has been discovered that certain blends of products can be used to efficiently coat the surface of hair fibers without resorting to the use of quaternary cationics: certain film-forming molecules with high molecular weights which dry to a flexible form can be formulated to strongly deposit onto the hair without using the electric charge as a driving force. We have noted that a blend of flexible film-forming compounds, when blended with quick-spreading emollients and adequately formulated into a fine emulsion, will deposit onto the hair and provide a significant conditioning effect. These compositions have been found to improve appearance and shine of the hair, improve manageability and allow for easy styling of the hair. Further, these formulations are considered to be safe toward the scalp as the components used are commonly used in skin care products and are considered harmless to the skin.

The choice of emollients is important: the role of the emollient two-fold: the chosen emollients spread rapidly and readily on the hair fibers and have a high affinity for the keratin and secondly, they solubilize the film-formers, reducing their viscosity and allowing a uniform deposition of this compound onto the hair surface. In this way, there is no need for a quaternary compound to adhere to the hair. Further, the higher molecular weights of the film formers will allow them to remain on the hair after rinsing which imparts a conditioning effect on the hair while their flexibility allows a natural movement of the hair.

Among the emollients that are useful in this application, we find the following:

Alkanes: these are also referred to as paraffinic substances and are emollients if the carbon content is between C12-C22—at these molecular weights, the products are liquid at room temperature and the lighter end of the range tends to spread more readily on hair and skin and feel less oily. We also consider squalane and hemisqualane to be useful emollients in this composition.

Esters: these are formed by linking an acid to an alcohol molecule, or a few acid molecules to a diol or polyol or else several alcohols to a polyacid. In general, the spreading is highest for molecules with a lighter molecular mass which generally refers to monoesters (one acid and one alcohol molecule) and these are considered the most useful in this application with a few exceptions that will be highlighted in the detailed list. Among the esters useful in this composition, we can list the following:

Ethyl myristate, ethyl palmitate, ethyl stearate, propyl myristate, propyl palmitate, propyl stearate, butyl myristate, butyl palmitate, butyl stearate, ethyl hexyl myristate, ethyl hexyl palmitate, ethyl hexyl stearate, isononyl isononanoate, glyceryl stearate, diethyl adipate, ethylhexyl benzoate, lauryl laurate, diethylhexylcarbonate, Diisostearyl Malate, Tricaprylyl Citrate, Isostearyl Isostearate, Isostearyl Palmitate, Myristyl Myristate, Decyl Oleate, Isocetyl Behenate, Isocetyl Myristate, Isocetyl Palmitate, Isocetyl Stearate, Isodecyl Oleate, Isopropyl Isostearate, Isopropyl Myristate, Isopropyl Palmitate, Methyl Acetyl Ricinoleate, Oleyl Oleate, Octydodecyl Erucate, Ethylhexyl Hydroxystearate, C₁₂₋₁₃ Alkyl Lactate, C₁₂₋₁₅ Alkyl Lactate, Cetyl Lactate, Isostearyl Lactate, Lauryl Lactate, Myristyl Lactate, Oleyl Lactate, Diethylhexyl Malate, Triisocetyl Citrate, Trioctyldodecyl Citrate, Diethylhexyl Maleate, C₁₂₋₁₅ Alkyl Benzoate, Dipropylene Glycol Dibenzoate, Dipropylene Glycol Benzoate, Cetyl Ethylhexanoate, Ethylhexyl Isononanoate,Isodecyl Ethylhexanoate, Isodecyl Isononanoate, Tridecyl Ethylhexanoate, Isotridecyl Isononanoate, Isostearyl Isononanoate, Cetearyl Isononanoate, Hexyl Decanoate, Cetearyl Ethylhexanoate, C₁₂₋₁₅ Alkyl Ethylhexanoate, Dicapryl Adipate, Dicaprylyl Maleate, Diisopropyl Dimer Dilinoleate, Diisopropyl Adipate, Diisobutyl Adipate, Diisopropyl Sebacate, Isodecyl Neopentanoate, Isostearyl Neopentanoate, Ethylhexyl Pelargonate, Myristyl Neopentanoate, Isostearyl Behenate, Octyldodecyl Neopentanoate, Tridecyl Neopentanoate, Diethyhexyl Succinate, neopentyl Glycol Dicaprate, Diethylene Glycol Diethylhexanoate, Propylene Glycol Myristyl Ether Acetate, Propylene Glycol Dicaprylate/Dicaprate, Neopentyl Glycol Diisostearate, Neopentyl Glycol Diethylhexanoate, Triisostearyl Citrate, hexyl laurate, Neopentyl Glycol Dicapelargonate, coco caprylate, dicaprylyl carbonate, coco-Caprylate/Caprate, Dicaprylyl Ether, Propylheptyl Caprylate, Cetearyl Isononanoate, decyl oleate.

In addition, certain light oils such as caprylic/capric triglyceride, glyceryl tripelargonate and ethers such as capryl ether caprate, coco-Caprylate/Caprate are useful as spreading emollients in the current invention.

The spreading emollients chosen from the list above are combined with film forming polymers such as:

Isostearyl Stearoyl Stearate, hydrogenated poly-isobutene, Trivent Mango Butter (Mangifera Indica (Mango) Seed Butter, acrylates/C12-22 Alkyl Methacrylate Copolymer, Butyl Ester of PVM/MA Copolymer, VP/Eicosene Copolymer, Ethyl Ester of PVM/MA Copolymer, Adipic Acid/Neopentyl Glycol/Trimellitic Anhydride Copolymer, VP/Hexadecene Copolymevr, Acrylates/Ethylhexyl Acrylate Copolymer, Acrylates Copolymer, Vinyl Caprolactam/VP/Dimethylaminoethyl Methacrylate Copolymer, Glyceryl Triacetyl Hydroxystearate, Polyhydroxystearic Acid, Algin, Sodium Acryloyldimethyl Taurate Copolymer, Vinyl Caprolactam/VP/Dimethylaminoethyl Methacrylate Copolymer, VP/VA Copolymer, Dilinoleic Acid/Propanediol Copolymer, Ethyl Ester of PVM/MA Copolymer, Methyl Methacrylate Crosspolymer, PVP, Acrylates/Dimethicone Copolymer, Norbornene/Tris (Trimethylsiloxy) silylnorbornene Copolymer, Butyl Acrylate/Isobornyl Methacrylate Copolymer, VP/Acrylates/Lauryl Methacrylate Copolymer, Polydiethyleneglycol Adipate/IPDI Copolymer, C28-52 Olefin/Undecylenic Acid Copolymer, Adipic Acid/Diglycol Crosspolymer, Ethylene/Propylene/Styrene Copolymer (and) Butylene/Ethylene/Styrene Copolymer, hydrogenated Dilinoleyl Hydrogenated Dimer Dilinoleate/Stearate, Chitosan Succinamide, Maleated Soybean Oil Glyceryl/Octyldodecanol Ester, Polyurethane-34, VA/Crotonates Copolymer, Triacontanyl PVP, Isobutylmethacrylate/Bis-Hydroxypropyl Dimethicone Acrylate Copolymer, Acrylates/Octylacrylamide Copolymer, Bis-Octyldodecyl Dimer Dilinoleate/Propanediol Copolymer, Polyester-7, Ammonium Acrylates Copolymer, Vinylamine/Vinyl Alcohol Copolymer, Ditrimethylolpropane Tetrastearate, Acrylates/Ethylhexyl Acrylate Copolymer, Styrene/Acrylates/Ammonium Methacrylate Copolymer, Polyester-5, VP/Eicosene Copolymer, VA/Crotonates/Vinyl Neodecanoate Copolymer, Butendiol/Vinyl Alcohol Copolymer, Polyester-10, Glyceryl Triacetyl Hydroxystearate, utyl Acrylate/Isopropylacrylamide/PEG-18 Dimethacrylate Crosspolymer, Acrylates/Lauryl Acrylate/Stearyl Acrylate/Ethylamine Oxide Methacrylate Copolymer, Vinyl Caprolactam/VP/Dimethylaminoethyl Methacrylate Copolymer, Acrylates/Steareth-20 Methacrylate Crosspolymer, Polyester-8, Polyurethane-14 (and) AMP-Acrylates Copolymer, Aluminum Starch Octenylsuccinate, Lauryl Methacrylate/Glycol Dimethacrylate Crosspolymer, VA/Butyl Maleate/Isobornyl Acrylate Copolymer, PPG-26/Dimer Dilinoleate Copolymer, Methacryloyl Ethyl Betaine/Acrylates Copolymer, Acrylates/C10-30 Alkyl Acrylate Crosspolymer, Polyester-37, VP/Eicosene Copolymer, Capryloyl Glycerin/Sebacic Acid Copolymer, Dimer Dilinoleyl Dimer Dilinoleate, Diisostearyl Dimer Dilinoleate, Diisopropyl Dimer Dilinoleate, Hydrogenated Castor Oil Dimer Dilinoleate, Triisostearoyl Polyglyceryl-3 Dimer Dilinoleate, diisostearoyl Polyglyceryl-3 Dimer Dilinoleate, Di-C20-40 Alkyl Dimer Dilinoleate, Hydrogenated Dilinoleyl Hydrogenated Dimer Dilinoleate/Stearate, Bis-Octyldodecyl Dimer Dilinoleate/Propanediol Copolymer, Phytosteryl/Isostearyl/Cetyl/Stearyl/Behenyl Dimer Dilinoleate, Mango Butter Dimer Dilinoleyl Esters/Dimer Dilinoleate Copolymer, Bis-Behenyl/Isostearyl/Phytosteryl Dimer Dilinoleyl Dimer Dilinoleate, Dilinoleic Acid/Propanediol Copolymer, Dilinoleamidopropyl Dimethylamine.

Among the polymers cited in the above list, only those that form a flexible film when dry are useful in this invention: polymers that dry down to a rigid/solid state and are used in styling products do not impart a natural, conditioned feel to the hair and are not useful in this application. Beyond the chemical description, the rigidity of a polymer is determined by its' molecular weight and the degree of cross-polymerization that it presents-we characterize the polymers as useful in this application if they present a liquid, gelatinous or viscous liquid appearance as individual raw materials-those that appear as solids when pure are not useful in this composition.

One or more emollients can be included in the hair care composition at a level of about 0.1% to about 25%, alternatively from about 1% to about 17% and alternatively from about 1.5% to about 9%, by weight of the hair care composition.

One or more film-forming polymers can be added in the hair care composition at a level of about 0.1% to about 10%, alternatively, from about 0.5% to about 5% and alternatively from about 0.25% to about 3.5%, by weight of the hair care composition. The film-forming polymers deposit on the surface of the hair fiber and provide improved shine, water repellency, and a reduced tendency to frizz.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be present in commercially available materials.

Herein “mixtures” is meant to include a simple combination of materials and any compounds that may result from their combination.

The term “molecular weight” as used herein refers to the average molecular weight.

The hair care composition comprises from about 0.1 wt % to about 30 wt. % of emollient or blend of emollients.

The hair care composition comprises from about 0.05 wt. % to about 10 wt. % of film-forming polymers.

The ratio of film forming agents to emollients can be comprised in the range of 2:1 to 1:50

The hair care composition comprises one or more high melting point fatty compounds. These can have a melting point of 25° C. or higher and can be selected from a group consisting of fatty alcohols, fatty acids, of fatty alcohols derivatives, fatty acid derivatives and mixtures thereof. The fatty alcohols useful in these compositions can have a carbon length of 14 to 30 carbon atoms and can be branched or linear.

In addition, other useful components such as pH modifiers, silicone compounds, plant extracts, panthenol and organic acids and preservatives and fragrances can be added to the composition as necessary. Exemplary silicone compounds include polydimethylsiloxane, polydiethylsiloxane and polymethylphenylsiloxane. Aminosilicones can also be added to the composition, especially the non quaternized aminosilicones which respect the non-quaternary compositions of the invention. Silicone copolymer emulsions are also useful in hair conditioning and may be added to the compositions in the disclosed invention.

In addition to film-formers, other polymers may also be included in compositions of the present invention as these can be useful in other ways. Some naturally derived polymers can be useful rheological stabilizers in hair conditioner compositions and can be included in the composition. Examples of these are guar gums, derivatized guar gums, derivatized cellulose, such as methyl cellulose, ethyl cellulose, hydroxyethyl cellulose, hydroxypropylmethyl cellulose, sodium carboxymethyl cellulose, crystalline cellulose, and carrageenan, xanthan and arabia gums, carob gum, karaya gum, pectin, agar, starch, algae colloids, dextran, succinoglucan, pulleran, starch-based polymers such as carboxymethyl starch, methylhydroxypropyl starch and alginic acid-based polymers. These can be useful to improve combing performance as well as acting as thickeners in the formulation. Synthetic polymers such as Carbomers (cross linked acrylic polymers), polyvinylpyrrolidone, polyvinyl alcohol can also be included in hair care compositions and are useful as rheological agents.

Hyaluronic acids and hyaluronic acid derivatives are also useful in hair care compositions and can be added in products covered by the present invention.

Emulsifiers can be included in certain embodiments of the hair care composition in order to help form a stable formulation. Examples of emulsifiers that can be used include alkoxylated fatty alcohols such as steareth-20, alkoxylated fatty acids, alkylpolyglucosides and glyceryl esters such as glyceryl stearate. In certain embodiments, emulsifiers amines can be included in about 0.1% to about 3.5% by weight in the composition.

Mono-alkyl amines are useful as emulsifiers and thickening agents and can be comprised in the compositions. Primary, secondary and tertiary fatty amines are useful in these compositions, particularly those comprising at least one alkyl chain of a carbon length of 12 to 22 carbon atoms.

In certain embodiments, certain tertiary amido amines can be included in about 0.1% to about 3.5% by weight in the composition; examples of such tertiary amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldimethylamine, palmitamidoethyldiethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldimethylamine, arachidamidoethyldiethylamine and diethylaminostearamide.

The composition is most often water-based formulation that may be prepared by dispersing all components (melted at a sufficient temperature if necessary) into an aqueous base under high shear (mixing). If high melting point ingredients are used, all the phases are heated to a temperature above the melting point (usually 70-85C) and ingredients are added and mixed until dispersed. Once adequately dispersed, the mixture is allowed to cool under continuous agitation until the mixture attains a temperature of 40C or less at which point the temperature-sensitive ingredients are added (usually preservatives, extracts and fragrance).

The hair care composition comprises a carrier liquid, preferably water, at a level of from about 50% to 96%, alternatively, from about 55% to about 85% by weight of the hair care composition. Accordingly, the hair care composition can be in the form of pourable liquids or sprayable liquids. Other water-miscible solvents and emollients, such as glycerol, butyl glycol or propanediol can be added to the aqueous phase to improve the softening and conditioning qualities of the composition.

In certain embodiments, the hair care composition can also comprise natural oils, such as sunflower seed oils, coconut oils, argan oil, jojoba oils and oils extracted from nuts or fruit kernels as well as palm oil, this list serving only to illustrate the nature of the oils and is not limitative. Alternatively, certain oils of natural derivation such as shea butter, cupuacu butter and jojoba esters can be usefully added to the hair care composition.

The invention further includes a method of conditioning hair, comprising applying a composition topically to the hair, wherein the quat-free composition comprises a mixture of emollients and flexible film-formers. The compositions of the present invention can be in the form of rinse-off products or leave-on products and can be formulated in a variety of product forms, including, but not limited to creams, gels, emulsions, mousses and sprays. The high compatibility of the composition with scalp and microbiome safety makes it particularly suitable for conditioning compositions, especially leave-on, quick rinse or no rinse compositions. These may be applied to dry hair, damp hair or wet hair. They may be rinsed off after application or allowed to remain on the hair after application which allows conditioning emollients to penetrate further into the hair fiber.

What is claimed is:

1. Hair care compositions free of quaternary ammonium compounds comprising:

4. from about 0.1% to about 25% in emollients, particularly those with viscosity at room temperature <5000cps and from about 0.05% to 10% of a flexible film-forming compound wherein the ratio of emollient to film-forming compound is from about 200:1 to about 1:1

2. The hair care composition of claim 1 wherein the ratio of emollient to flexible film-forming compound is from about 20:1 to about 2:1

3. The hair care composition of claim 1. wherein the composition comprises from about 2% to 15% emollients and from about 0.3% to 7% of a flexible film-forming agent.

4. The hair care composition of claim 1 wherein the emollient is one or more of the following: Ethyl myristate, ethyl palmitate, ethyl stearate, propyl myristate, propyl palmitate, propyl stearate, butyl myristate, butyl palmitate, butyl stearate, ethyl hexyl myristate, ethyl hexyl palmitate, ethyl hexyl stearate, isononyl isononanoate, glyceryl stearate, diethyl adipate, ethylhexyl benzoate, lauryl laurate, diethylhexylcarbonate, Diisostearyl Malate, Tricaprylyl Citrate, Isostearyl Isostearate, Isostearyl Palmitate, Myristyl Myristate, Decyl Oleate, Isocetyl Behenate, Isocetyl Myristate, Isocetyl Palmitate, Isocetyl Stearate, Isodecyl Oleate, Isopropyl Isostearate, Isopropyl Myristate, Isopropyl Palmitate, Methyl Acetyl Ricinoleate, Oleyl Oleate, Octydodecyl Erucate, Ethylhexyl Hydroxystearate, C₁₂₋₁₃ Alkyl Lactate, C₁₂₋₁₅ Alkyl Lactate, Cetyl Lactate, Isostearyl Lactate, Lauryl Lactate, Myristyl Lactate, Oleyl Lactate, Diethylhexyl Malate, Triisocetyl Citrate, Trioctyldodecyl Citrate, Diethylhexyl Maleate, C₁₂₋₁₅ Alkyl Benzoate, Dipropylene Glycol Dibenzoate, Dipropylene Glycol Benzoate, Cetyl Ethylhexanoate, Ethylhexyl Isononanoate, Isodecyl Ethylhexanoate, Isodecyl Isononanoate, Tridecyl Ethylhexanoate, Isotridecyl Isononanoate, Isostearyl Isononanoate, Cetearyl Isononanoate, Hexyl Decanoate, Cetearyl Ethylhexanoate, C₁₂₋₁₅ Alkyl Ethylhexanoate, Dicapryl Adipate, Dicaprylyl Maleate, Diisopropyl Dimer Dilinoleate, Diisopropyl Adipate, Diisobutyl Adipate, Diisopropyl Sebacate, Isodecyl Neopentanoate, Isostearyl Neopentanoate, Ethylhexyl Pelargonate, Myristyl Neopentanoate, Isostearyl Behenate, Octyldodecyl Neopentanoate, Tridecyl Neopentanoate, Diethyhexyl Succinate, neopentyl Glycol Dicaprate, Diethylene Glycol Diethylhexanoate, Propylene Glycol Myristyl Ether Acetate, Propylene Glycol Dicaprylate/Dicaprate, Neopentyl Glycol Diisostearate, Neopentyl Glycol Diethylhexanoate, Triisostearyl Citrate, hexyl laurate, Neopentyl Glycol Dicapelargonate, coco caprylate, dicaprylyl carbonate, coco-Caprylate/Caprate, Dicaprylyl Ether, Propylheptyl Caprylate, Cetearyl Isononanoate, decyl oleate, capryl caprate triglyceride, glyceryl tripelargonate, capryl ether caprate and/or coco-Caprylate/Caprate, squalane or hemisqualane.

5. The hair care composition of claim 1 wherein the flexible film-forming agent is one or a blend of the following compounds: Isostearyl Stearoyl Stearate, hydrogenated poly-isobutene, Trivent Mango Butter (Mangifera Indica (Mango) Seed Butter, acrylates/C12-22 Alkyl Methacrylate Copolymer, Butyl Ester of PVM/MA Copolymer, VP/Eicosene Copolymer, Ethyl Ester of PVM/MA Copolymer, Adipic Acid/Neopentyl Glycol/Trimellitic Anhydride Copolymer, VP/Hexadecene Copolymevr, Acrylates/Ethylhexyl Acrylate Copolymer, Acrylates Copolymer, Vinyl Caprolactam/VP/Dimethylaminoethyl Methacrylate Copolymer, Glyceryl Triacetyl Hydroxystearate, Polyhydroxystearic Acid, Algin, Sodium Acryloyldimethyl Taurate Copolymer, Vinyl Caprolactam/VP/Dimethylaminoethyl Methacrylate Copolymer, VP/VA Copolymer, Dilinoleic Acid/Propanediol Copolymer, Ethyl Ester of PVM/MA Copolymer, Methyl Methacrylate Crosspolymer, PVP, Acrylates/Dimethicone Copolymer, Norbornene/Tris (Trimethylsiloxy) silylnorbornene Copolymer, Butyl Acrylate/Isobornyl Methacrylate Copolymer, VP/Acrylates/Lauryl Methacrylate Copolymer, Polydiethyleneglycol Adipate/IPDI Copolymer, C28-52 Olefin/Undecylenic Acid Copolymer, Adipic Acid/Diglycol Crosspolymer, Ethylene/Propylene/Styrene Copolymer (and) Butylene/Ethylene/Styrene Copolymer, hydrogenated Dilinoleyl Hydrogenated Dimer Dilinoleate/Stearate, Chitosan Succinamide, Maleated Soybean Oil Glyceryl/Octyldodecanol Ester, Polyurethane-34, VA/Crotonates Copolymer, Triacontanyl PVP, Isobutylmethacrylate/Bis-Hydroxypropyl Dimethicone Acrylate Copolymer, Acrylates/Octylacrylamide Copolymer, Bis-Octyldodecyl Dimer Dilinoleate/Propanediol Copolymer, Polyester-7, Ammonium Acrylates Copolymer, Vinylamine/Vinyl Alcohol Copolymer, Ditrimethylolpropane Tetrastearate, Acrylates/Ethylhexyl Acrylate Copolymer, Styrene/Acrylates/Ammonium Methacrylate Copolymer, Polyester-5, VP/Eicosene Copolymer, VA/Crotonates/Vinyl Neodecanoate Copolymer, Butendiol/Vinyl Alcohol Copolymer, Polyester-10, Glyceryl Triacetyl Hydroxystearate, utyl Acrylate/Isopropylacrylamide/PEG-18 Dimethacrylate Crosspolymer, Acrylates/Lauryl Acrylate/Stearyl Acrylate/Ethylamine Oxide Methacrylate Copolymer, Vinyl Caprolactam/VP/Dimethylaminoethyl Methacrylate Copolymer, Acrylates/Steareth-20 Methacrylate Crosspolymer, Polyester-8, Polyurethane-14 (and) AMP-Acrylates Copolymer, Aluminum Starch Octenylsuccinate, Lauryl Methacrylate/Glycol Dimethacrylate Crosspolymer, VA/Butyl Maleate/Isobornyl Acrylate Copolymer, PPG-26/Dimer Dilinoleate Copolymer, Methacryloyl Ethyl Betaine/Acrylates Copolymer, Acrylates/C10-30 Alkyl Acrylate Crosspolymer, Polyester-37, VP/Eicosene Copolymer, Capryloyl Glycerin/Sebacic Acid Copolymer, Dimer Dilinoleyl Dimer Dilinoleate, Diisostearyl Dimer Dilinoleate, Diisopropyl Dimer Dilinoleate, Hydrogenated Castor Oil Dimer Dilinoleate, Triisostearoyl Polyglyceryl-3 Dimer Dilinoleate, diisostearoyl Polyglyceryl-3 Dimer Dilinoleate, Di-C20-40 Alkyl Dimer Dilinoleate, Hydrogenated Dilinoleyl Hydrogenated Dimer Dilinoleate/Stearate, Bis-Octyldodecyl Dimer Dilinoleate/Propanediol Copolymer, Phytosteryl/Isostearyl/Cetyl/Stearyl/Behenyl Dimer Dilinoleate, Mango Butter Dimer Dilinoleyl Esters/Dimer Dilinoleate Copolymer, Bis-Behenyl/Isostearyl/Phytosteryl Dimer Dilinoleyl Dimer Dilinoleate, Dilinoleic Acid/Propanediol Copolymer and/or Dilinoleamidopropyl Dimethylamine.

6. The hair care composition of 1, further comprising a silicone compound.

7. The hair care composition of claim 1 wherein the hair care composition is a hair conditioner.

8. The hair care composition of claim 1 wherein the hair care composition is a leave-in hair conditioner.

9. The hair care composition of claim 1 wherein the hair care composition is a rinse-off hair conditioner.

10. The hair care composition of claim 1 wherein the hair care composition is a hair conditioning spray.

11. The hair care composition of claim 1 wherein the hair care composition is a hair conditioning cream.

12. The hair care composition of claim 1 wherein the hair care composition comprises a natural oil.

13. The conditioner of claim 1 further comprising an additional ingredient selected from extracts useful for providing fragrance, amino acids, nucleic acids, vitamins, hydrolyzed proteins and derivatives thereof, glycerin and derivatives thereof, microbiocides, antifungals, antiseptics, antioxidants, UV absorbers, dyes and pigments, chelating agents, pH balancing agents, moisturizers and mixtures thereof.

14. The conditioner of claim 1 further comprising an additional ingredient selected from EDTA, glutamic acid, glycerin, panthenol, stearyl alcohol, cetyl alcohol, cyclomethicone, dimethicone, pH adjustment additives, and mixtures thereof.

15. A method of conditioning hair comprising applying to the hair the composition of claim 1.
